(19) United States
(12) Patent Application Publication
(10) Pub. No.: US 2022/0223266 A1
(43) Pub. Date: Jul. 14, 2022

(54) System, Method, and Apparatus for Staff or Resource Deployment

(71) Applicant: Ways Investments, LLC, Sarasota, FL (US)

(72) Inventor: Mark Edward Gray, Lakewod Ranch, FL (US)

(73) Assignee: Ways Investments, LLC, Sarasota, FL (US)

(21) Appl. No.: 17/559,210

(22) Filed: Dec. 22, 2021

Related U.S. Application Data
(60) Provisional application No. 63/136,266, filed on Jan. 12, 2021.

(51) Int. Cl. G16H 40/20; G16H 50/30; H04L 67/12

(52) U.S. Cl. CPC G16H 40/20; H04L 67/12; G16H 50/30

(57) ABSTRACT

A method of healthcare allocation includes receiving, at an artificial intelligence engine, health data from sensors over a period of time. The sensors read the health data of a plurality of patients. Thereby, as the artificial intelligence engine receives the health data, the artificial intelligence engine learns a baseline health assessment of each of the patients. After the period of time elapses, the artificial intelligence engine continuously receives the health data from the sensors. If the health data singularly or in combination indicates an absolute healthcare issue exists, the artificial intelligence engine allocates/recommends at least one resource to one of the patients that is associated with the health data. Periodically, the artificial intelligence engine scans all patients and generates a priority for help for each of the patients and then allocates/recommends at least one resource to each patient in a subset of the patients based upon the priority.

### CROSS-REFERENCE TO RELATED APPLICATION

This application claims the benefit of U.S. provisional application No. 63/136,266 filed on Jan. 12, 2021, the disclosure of which is incorporated by reference.

### FIELD

This invention relates to the fields of healthcare and more particularly to a system or method for prioritizing patient care for those in most need.

### BACKGROUND

As the population of many countries ages, more and more people are reaching an age where independent life becomes difficult and, often, such people require varying levels of assistance to survive and be comfortable. Such assistance includes home care visits for those who can mostly care for themselves with a little assistance, as well as institutional care where for those who cannot manage day-to-day life without significant help.

As the percentage of a country's population shifts to greater numbers of people requiring some level of care, that portion of the population becomes a tax on the remaining part of the population, requiring more and more people enter care-giving careers. At some point, such a country will fail to exist if every working individual of that country is providing care to the aging, as there will be little or no remaining workforce available to provide other services, manufacturing, farming, food distribution, etc.

It is then evident that, in the future, allocation of precious healthcare workers will be critical, making sure these precious resources are utilized efficiently where the highest needs exist.

In today's environment, allocation of healthcare workers is typically on a demand or a scheduled basis. For example, a 7:00 AM, every day, a healthcare worker visits a certain patient to take vitals and administer a medication or when a patient operates a call button, a healthcare worker travels to the patient's room to discover what is needed. In these situations, there is no priority given to a patient that needs attention more than one that is stable and there is no consideration to the travel time between patients.

What is needed is a system and method that constantly monitors a population of patients, learns normal readings, and monitors deviations from the normal readings so as to efficiently assign healthcare workers and resources.

### SUMMARY

The healthcare allocation system utilizes artificial intelligence to monitor the health of a population of patients by way of automatic and/or manual health measurements. An artificial intelligence engine receives health data for each of the patients in the population and learns a baseline health for each patient in the population. The artificial intelligence engine then continuously monitors health data for each of the patients in the population and periodically recommends, refers, or assigns healthcare resources based upon a differential from each patient's baseline health measurements or based upon an absolute health measurement. As an example of an absolute health measurement, if one of the patients in the population has a health measurement that indicates a significant issue such as an oxygen level less than 80%, that patient has an absolute healthcare issue and that patient is assigned as a priority for healthcare resources (e.g., a visit from a nurse or doctor). As an example of a differential measurement, if one patient has a normal heart rate of 40 beats per minute and their current heart rate is 40 beats per minute, this matches the baseline health measurements and, therefore, no healthcare resources are assigned. On the other hand, if that same patient has a normal heart rate of 60 beats per minute and their current heart rate is 40 beats per minute, this is significantly less than the baseline health measurements and, therefore, healthcare resources are recommended, referred, or assigned as a priority depending upon all other patient's needs and associated priority. Both staff (e.g., doctors, nurses, technicians, support staff, drivers); and equipment (e.g., ambulances, ventilators, dialysis, medications); and even certain medications that are scarce are considered resources that are recommended/assigned by the disclosed healthcare allocation system.

In one embodiment, a method of healthcare allocation is disclosed. The method includes receiving, at an artificial intelligence engine, health data from sensors over a period. The sensors read the health data of a plurality of users or patients. Thereby, as the artificial intelligence engine receives the health data, the artificial intelligence engine learns a baseline health assessment of each of the users. After the period of time elapses, the artificial intelligence engine continuously receives the health data from the sensors. If the health data singularly or in combination indicates an absolute healthcare issue exists, the artificial intelligence engine allocates at least one resource to one of the users that is associated with the health data. Periodically, the artificial intelligence engine scans all users and generates a priority for help for each of the users and then allocates at least one resource to a subset of the users based upon the priority.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a resource allocation system.

FIG. 2 illustrates an exemplary computer as used in the resource allocation system.

FIG. 3 illustrates a learning mode of the resource allocation system.

FIG. 4 illustrates a usage mode of the resource allocation system.

FIG. 5 illustrates an exemplary three-input feed forward neural network having two hidden neurons.

FIG. 6 illustrates an exemplary program flow during a learning mode of the resource allocation system.

FIGS. 7-9 illustrate exemplary program flows during the predictive process of the resource allocation system.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

Throughout this description the terms user and patient are used interchangeably to indicate a being or person that is monitored by the resource allocation system.

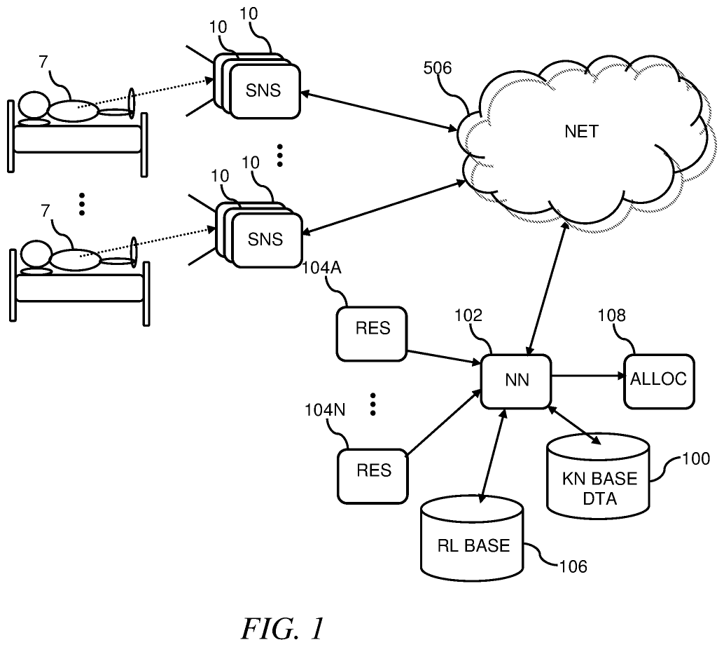

Referring to FIG. 1, an exemplary data connection diagram of the resource allocation system is shown. In the example shown, inputs from a multitude of sensors 10 are fed to a neural network 102 (e.g., artificial intelligence engine). The sensors 10 are associated with patients 7 (e.g., users) on a one-to-one or several-to-one based, in which, each patient 7 is monitored by at least one sensor 10. The sensors 10 are either invasive or non-invasive. For example, there exist sensor arrays that measure certain healthcare information of a patient 7 from distances of up to three feet, not requiring contact with the patient 7. Cameras and microphones are non-invasive. More invasive sensors 10 are also anticipated such as blood pressure cuffs, finger-worn oxygen level sensors, step counters, etc., that must be worn/carried.

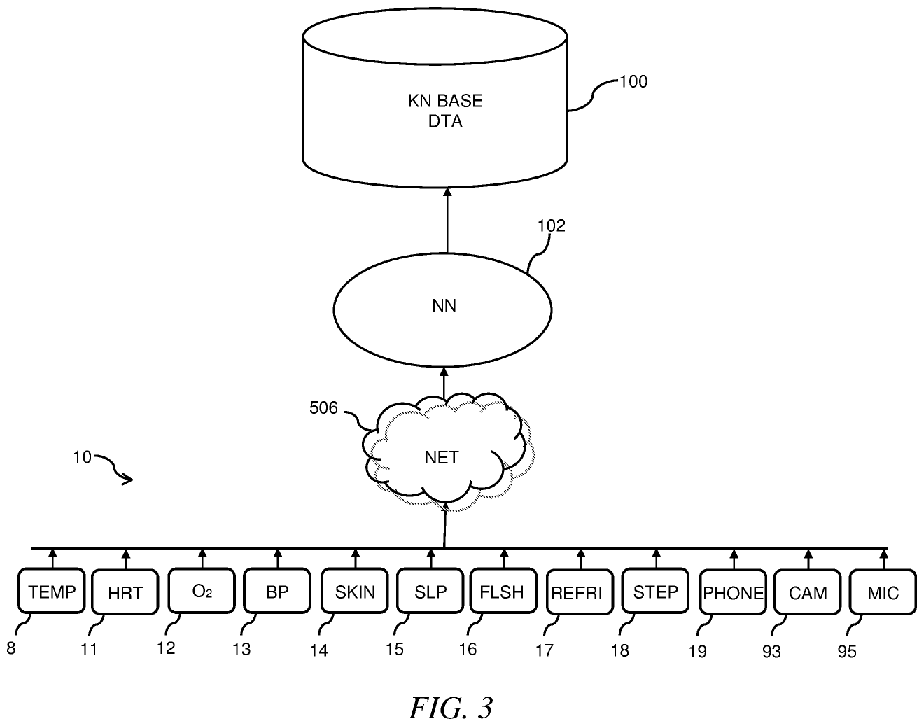

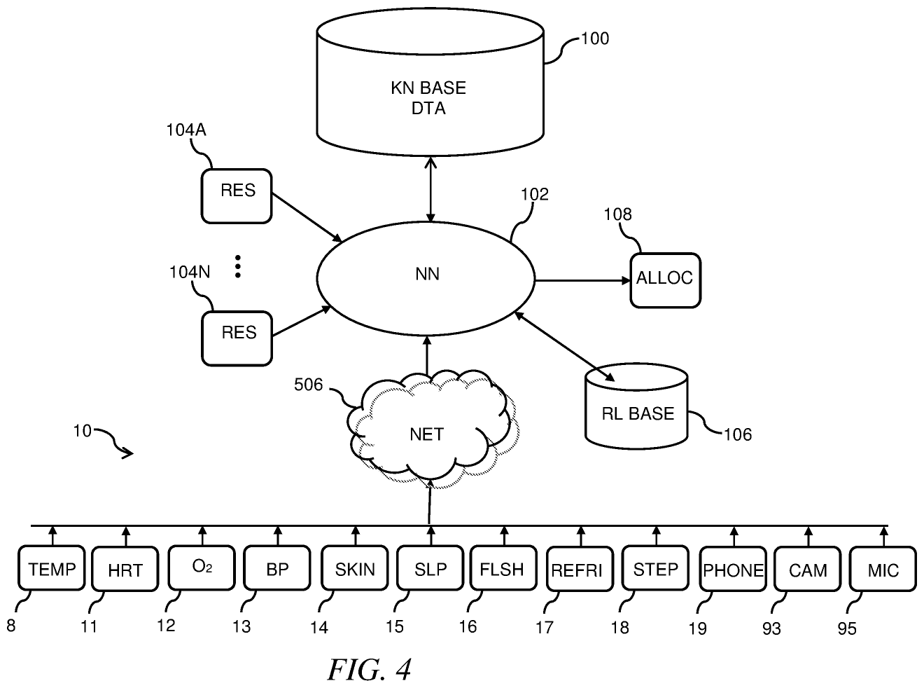

Although the inputs from the sensors 10 are shown passing through a network 506 (e.g., a local area network, a wide area network, wired or wireless), a direct connection is equally anticipated. Examples of such sensors 10 as shown in FIGS. 3 and 4 include health sensors such as temperature, heart rate, oxygen levels, blood pressures; and any other sensor that indicates certain activities such as camera inputs, microphone inputs, inputs from cell phones, door sensors (e.g., related to refrigerator or cabinet access), bathroom flush sensors, etc. In some embodiments, the microphone or any device having a microphone such as a smart speaker, monitors the environment and recognizes certain sounds such as a toilet flush, a cabinet door opening, a drawer opening/closing, or a refrigerator access and provides health data regarding such activities. The artificial intelligence engine, after learning behavior of the patients 7, will monitor norms of such activities and determine differential situations such as the patient 7 using the toilet significantly more than normal or not using the toilet for an extended period of time. The artificial intelligence engine also determines when significant periods of inactivity are detected, possibly indicating the patient 7 is in stress or has died.

Sets of the sensors 10 are associated with a patient 7 and there are many patients 7. In some embodiments, the sensors 10 are associated with a location at which the patient 7 is expected such as a hospital room, care facility room, home, etc. Periodically or continuously, the sensors 10 provided data regarding each of the patients 7 related to the current health of the patient and activities of the patient 7 such as steps taken, bathroom visits, kitchen visits, etc. The health data from the sensors 10 feed the neural network 102, which learns the normal health for each patient. Initially, the neural network 102 learns from the health data regarding each of the patients 7, storing data and inferences in a knowledge base 100. After substantial knowledge is acquired and stored in the knowledge base 100, the neural network 102 monitors the health data regarding the patients 7 to detect any possible abnormalities that indicate help is needed for that patient 7, then with inputs from the rule base 106, the neural network 102 determines the set of patients 7 that currently need help and prioritizes allocation of the resources 104A-104N (e.g. staff, equipment, etc.) to at least a subset of the patients 7 based upon the rule base 106. The rule base 106 includes rules for allocating the resources 104A-104N based upon absolute health assessments and/or differential health assessments. For example, a rule in the rule base 106 indicates that if a temperature of a patient 7 is over 105° F. then an allocation value for a particular resource to that patient 7 is high. The resource is, for example, a nurse if the patient 7 is in a hospital environment. If the patient 7 is not in a hospital environment, then the resource is, for example, a healthcare worker that will call the patient 7 or call an ambulance, etc. Another example or a rule in the rule base 106 is a heart rate of a patient 7 is 25% greater than normal, than an allocation value for a particular resource (e.g., cardiac doctor) to that patient 7 is medium.

Periodically, the resources 104A-104N are allocated to the patients 7 based upon the current health-index values for all patients 7. For example, assume that the resources 104A-104N are nurses in a hospital (note that there are many types of resources 104A-104N anticipated such as doctors, medical staff, hospital rooms, vehicles such as ambulances, emergency medical technicians, police, fire, non-medical staff), and medications, especially scarce medications. Periodically, the resource allocation system scans the allocation values for a resource 104A-104N, for example nurses, allocating the nurses to the patients 7 having the highest allocation values first (e.g., high health-index values indicating poor health). After the patients 7 having the highest health-index values are assigned resources 104A-104N, patients 7 having the next lower health-index values are assigned resources 104A-104N, etc. Note that any granularity of allocation values is anticipated such as high/medium/low, to numeric arranges (e.g., 1-100), etc.

In some embodiments, physical access and/or resource skills are considered in assigning the resources, in that, a resource 104A-104N (e.g., nurse) having infection experience is assigned to the patient 7 having a high temperature and a resource 104A-104N having cardiac experience is assigned to the patient 7 having an elevated heart rate or arrythmia. Further, it is anticipated that each resource 104A-104N has a location and a service area such as “general hospital, 3^{rd} floor only” and that resource is only assigned to patients 7 on the 3^{rd} floor. It is further anticipated that the service area has a normal value and an emergency value such as “3^{rd} floor only, 4th and 5th floor in emergencies.” In this way, if there is no resource 104A-104N available for a patient 7 with a high health-index value, but on the 5^{th} floor, this resource 104A-104N is available for allocation.

Once an allocation is made, an allocation module 108 processes the allocations by alerting each resource 104A-104N of the patient 7 that is in need of help. The allocation module 108 communicates with existing systems to emit alerts to resource 104A-104N (e.g., nurses, staff), interfaces with devices carried by resource 104A-104N such as smartphones, interfaces with call centers for help escalation, etc. In the latter, the call centers receive alerts such as “movement from patient 7 has not been detected for 12 hours” and performs escalation. In some embodiments, the call centers have demographic, medical and/or historical data regarding each patient 7 and attempt to reach out to the patient 7 and/or care givers for the patient 7 and, if needed, agents at the call center will escalate to summon emergency services for the patient 7 such as ambulances, police, fire, etc.

In some embodiments, the actual allocations of resources 104A-104N are made by a person after receiving suggested allocations from the resource allocation system. For example, the resource allocation system will suggest to allocate one of the resources 104A-104N to a particular patient 7 and an administrative person will make the final call as to whether to assign that resource, assign a different resource, or not assign any resource to that particular patient.

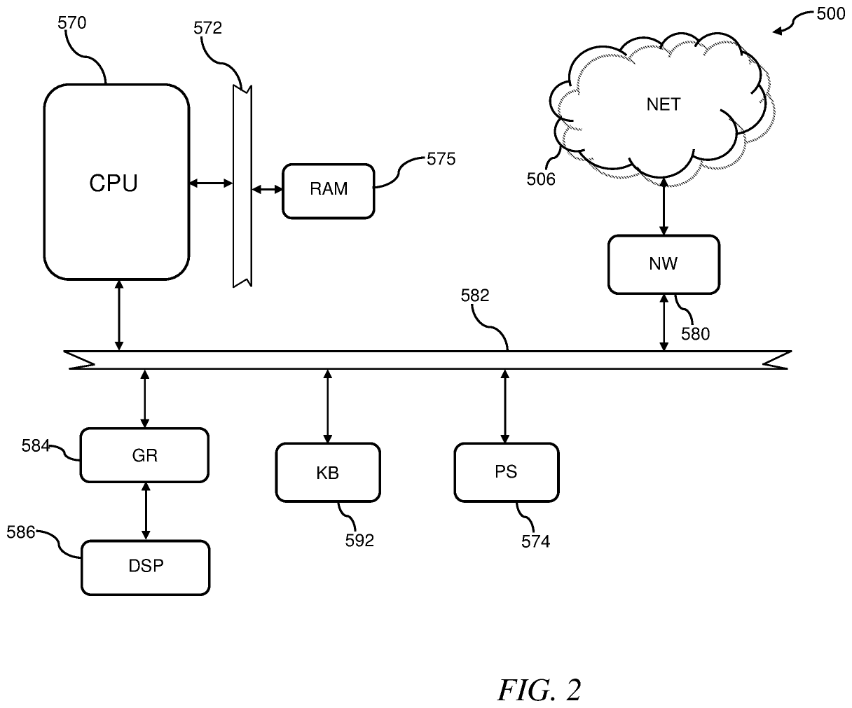

Referring to FIG. 2, a schematic view of a typical computer system 500 as used in the resource allocation system is shown. This exemplary server computer system 500 is shown in its simplest form. Different architectures are known that accomplish similar results in a similar fashion and the present invention is not limited in any way to any particular computer system architecture or implementation. In this exemplary computer system 500, a processor 570 executes or runs programs in a random-access memory 575. The programs are generally stored within a persistent memory 574 and loaded into the random-access memory 575 when needed. The processor 570 is any processor, typically a processor designed for computer systems with any number of core processing elements, etc. The random-access memory 575 is connected to the processor by, for example, a memory bus 572. The random-access memory 575 is any memory suitable for connection and operation with the selected processor 570, such as SRAM, DRAM, SDRAM, RDRAM, DDR, DDR-2, etc. The persistent memory 574 is any type, configuration, capacity of memory suitable for persistently storing data, for example, magnetic storage, flash memory, read only memory, battery-backed memory, magnetic memory, etc. The persistent memory 574 is typically interfaced to the processor 570 through a system bus 582, or any other interface as known in the industry.

Also shown connected to the system bus 582 is a network interface 580 (e.g., for connecting to the network 506), a graphics adapter 584 and a keyboard interface 592 (e.g., Universal Serial Bus—USB). The graphics adapter 584 receives information from the processor 570 and controls what is depicted on a display 586. The keyboard interface 592 provides navigation, data entry, and selection features.

In general, some portion of the persistent memory 574 is used to store programs, executable code, data, and other data, etc.

The peripherals are examples and other devices are known in the industry such as pointing devices, touch-screen interfaces, speakers, microphones, USB interfaces, Bluetooth transceivers, Wi-Fi transceivers, image sensors, temperature sensors, etc., the details of which are not shown for brevity and clarity reasons.

The resource allocation system is anticipated to be implemented in hardware, software, or any combination thereof.

Referring to FIG. 3, the resource allocation system learns baseline health information about the patients 7 though any types of sensors 10, including, but not limited to, temperature sensors 8, heart rate sensors 11, oxygen sensors 12, blood pressure sensors 13, skin color sensors 14, sleep sensors 15, toilet flush sensors 16, refrigerator opening sensors 17, pedometers 18, phone input sensors 19, cameras 93, microphones 95, etc. During initial operation, the neural network 102 of the resource allocation system receives inputs from the sensors 10 associated with many patients. As there are anticipated tens of sensors 10 per patient 7 and thousands of patients, the neural network 102 processes inputs from tens of thousands of sensors 10 to develop a knowledge base 100 having neural network inputs for each of the patients 7.

Referring to FIG. 4, after learning is complete, the resource allocation system monitors health data for the patients 7 though any or all of the above noted types of sensors 10, including, but not limited to, temperature sensors 8, heart rate sensors 11, oxygen sensors 12, blood pressure sensors 13, skin color sensors 14, sleep sensors 15, toilet flush sensors 16, refrigerator opening sensors 17, pedometers 18, phone input sensors 19, cameras 93, microphones 95, etc. During operation, the neural network 102 of the resource allocation system receives inputs from the sensors 10 associated with many patients 7. As there are anticipated tens of sensors 10 per patient 7 and thousands of patients 7, the neural network 102 processes inputs from tens of thousands of sensors 10 and updates the neural network inputs for each of the patients 7 based upon current measurements.

As data is received from the sensors 10 and processed by the neural network 102, rules from the rule base 106 work in the neural network 102 to generate allocation values for each patient 7 that is in need of help, then to allocate the resources 104A-104N to the patients 7 that are in need of help based upon the allocation values for each patient 7 and the availability and scope of each of the resources 104A-104N.

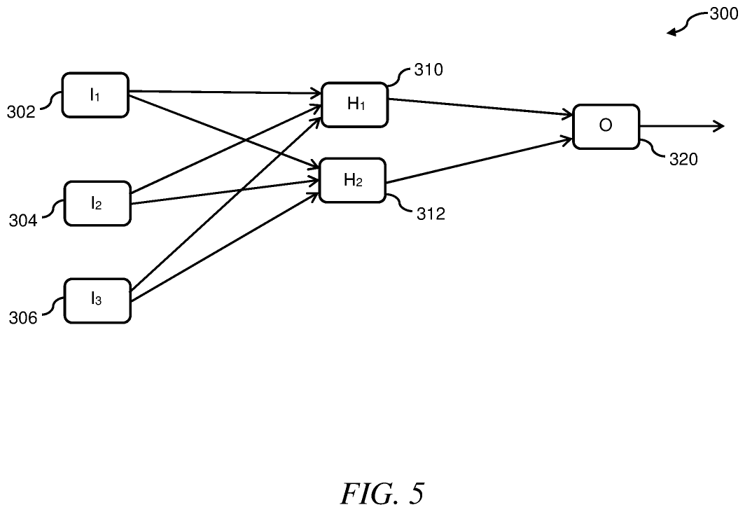

A sample cell of a neural network 102 of the resource allocation system is shown in FIG. 5. A mathematical function is trained using a first set of inputs 302/304/306 such that subsequent inputs 302/304/306 of the first mathematical function when applied to a second mathematical function 310/320 enable the second mathematical function to process a second set of inputs 302/304/306 producing a value indicative of differences or changes between the sets of inputs 302/304/306. One such mathematical function suitable for this purpose is that of the Neural Network taken from the science of Artificial Intelligence.

Referring to FIG. 5, an exemplary implementation of the 302/304/306 within which a mathematical process 300 represented by a simplified multilayer feed forward neural network is depicted. During a learning process, iterative sampling of sensors 8/11/12/13/14/15/16/17/18/19/20/93/95, etc., are processed by the neural network in training mode over a period of sufficient duration to, in effect, learn the baseline sensory input values for each patient 7. For each iteration, input values are fed into 302, 304 and 306 neurons with adjustments being made to weights and biases of hidden neurons 310 and 312 based on deviations between the output value of neuron 320 and desired sample output. The iterative process is repeated using newly captured sensory inputs with continued refinements by use of error function feedbacks being applied to hidden neuron weights and biases. After the multi-iteration cycle, the accumulated hidden neuron weights and biases are saved to a knowledge base as a dataset aligned to time such that the collection of saved datasets represents a timeline of sensory sampling events. During a subsequent predictive process newly acquired sensory inputs are fed into input neurons 302, 304 and 306 of a neural network that was provisioned with a dataset of weights and biases taken from the knowledge base timeline relative to the same time period with the resulting output value from neuron 320 representing a value between 0 and 1 that represents the probability the newly acquired sensory inputs are like or similar to the original set of sensory inputs employed to learn and create the knowledge dataset.

It is fully anticipated that the knowledge base 100 be organized to compartmentalize health-index data by various parameters such as time-of-day, day-of-week, visitation schedules, dining schedules, etc., as health-indexes will vary during sleep, weekends, lunch/dinner, etc.

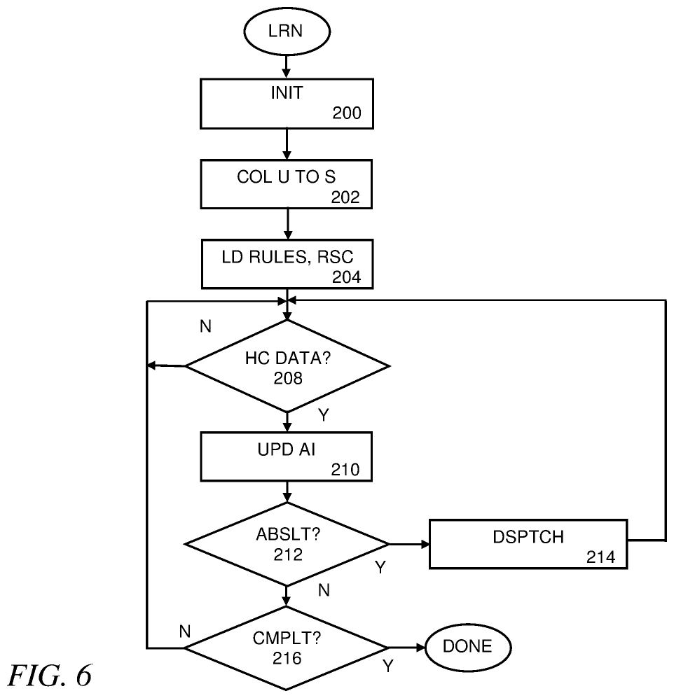

Referring to FIG. 6, an exemplary program flow indicative of training and learning mode of the resource allocation system is shown. The training and learning mode begins with an initialization step 200 which, among other things, initializes the knowledge base 100. In some embodiments, this initialization process includes collecting 202 associations between each user (patient 7) of the resource allocation system and one or more sensors 10 (e.g., informing resource allocation system which sensors 10 acquire health data for which patient 7). In some embodiments, a patient location is the focus of this correlation, for example, the patient's room, bathroom, of bed where the sensors are located. These associations between patients 7 and sensors 10 are needed for correlating input data from the sensors 10 to individual patients 7. The rules for allocating (or recommending) resources 104A-104N and the resource identifications of the resources 104A-104N are anticipated to be stored in any format and location. In this example, the rules and resource identifications are loaded 204 into the resource allocation system. The resource identifications identify each of the resources 104A-104N as to the type of the resource, location of the resources, service area of the resource, and any other attribute of the resource such as latency and reliability.

The training and learning phase 208/210/212 are anticipated to operate before full operation of the resource allocation system as well as during normal operation of the resource allocation system. Therefore, the resource allocation system constantly learns by assimilation of current inputs. As health data are captured 208 from the sensors 10, the health data is processed 210 (e.g., by use of mathematical process 300). The duration of training and learning is a function of the type of health data being learned. In some embodiments, the training and learning phase operates for a period of time (e.g., several days, a week, several weeks), collecting health data and loading the artificial intelligence engine until sufficient health data has been collected at which time the training and learning phase is complete 216.

As discussed above, the AI engine will make at least two types of assessments for each patient 7: absolute health assessments and differential health assessments. During the learning phase and until sufficient amounts of health data are collected for each patient 7, it will be error-prone for the AI engine to make proper differential health assessments (e.g., one patient 7 has a heart rate that is 20% higher than normal) as the AI engine has not yet established baselines for each patient 7. On the other hand, even though during the training and learning phase, differential health assessments are typically suppressed, it is anticipated that absolute health assessments be enabled (e.g., one patient 7 has an oxygen level below 80% or a heart rate lower than 50 beats per minute). In such, if any absolute health assessment threshold (or combination of thresholds) is detected 212, one or more of the resources 104A-104N are recommended/allocated/dispatched 214. By combination, it is anticipated that the rules include combination rules as well. For example, an oxygen level of 90% alone does not trigger an absolute health assessment and a heart rate of 81 beats per minute alone does not trigger an absolute health assessment, but an oxygen level of 90% coupled with a heart rate of over 81 beats per minute triggers an absolute health assessment.

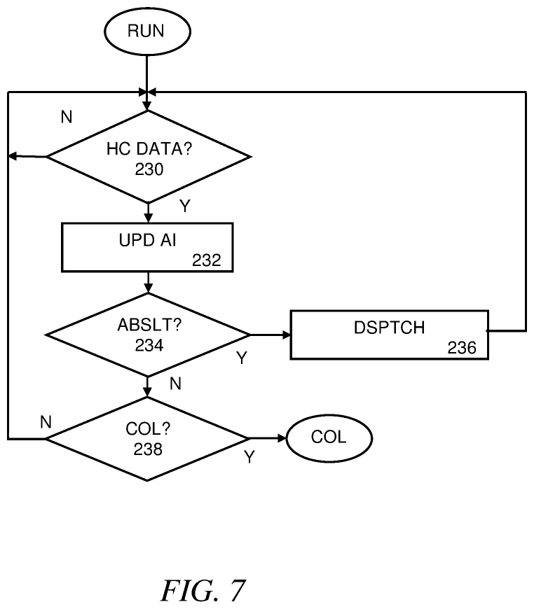

Referring to FIG. 7, an exemplary program flow indicative of normal operation of the resource allocation system is shown. The “Run” mode is a loop that checks to see if health data is received 230. Note that it is fully anticipated that each time through the loop, bulk health data be received (e.g., health data from a plurality of sensors 10 for one or more patients 7) or individual healthcare datum from a single sensor 10. For the sake of brevity, the example shown in FIG. 7 will process individual healthcare datum through each iteration of the loop 230/232/234/236/238.

As health data are captured 230 (e.g., from one or more the sensors 10), the health data is processed 232 (e.g., by use of mathematical process 300) to continuously update the artificial intelligence engine and, therefore, the knowledge base 100.

As discussed above, the AI engine will make at least two types of assessments for each patient 7: absolute health assessments and differential health assessments. During the learning phase and until sufficient amounts of health data are collected for each patient 7, it will be error-prone for the AI engine to make proper differential health assessments (e.g., one patient 7 has a heart rate that is 20% higher than normal) as the AI engine has not yet established baselines for each patient 7. On the other hand, even though during the training and learning phase, differential health assessments are typically suppressed, it is anticipated that absolute health assessments be enabled (e.g., one patient 7 has an oxygen level below 80% or a heart rate lower than 50 beats per minute). In such, if any absolute health assessment threshold (or combination of thresholds) is detected 234 during the “Run” mode, one or more of the resources 104A-104N are recommended/allocated/dispatched 236. By combination, it is anticipated that the rules include combination rules as well. For example, an oxygen level of 90% alone does not trigger an absolute health assessment and a heart rate of 81 peats per minute alone does not trigger an absolute health assessment, but an oxygen level of 90% coupled with a heart rate of over 81 beats per minute triggers an absolute health assessment.

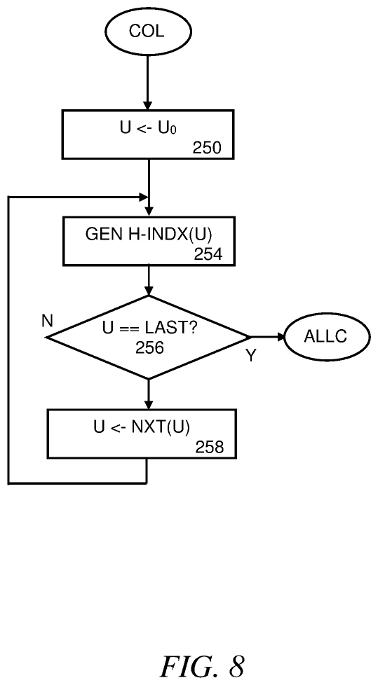

Periodically, it is determined that it is time to collate. The step of collating involves processing the more recent data collected for each patient 7 and determining if any of the resources 104A-104N need be recommended/allocated/dispatched to any of the patients 7. Therefore, if it is time to collate 238, the collate process of FIG. 8 is performed, otherwise the loop 230/232/234/236/238 iterates.

Referring to FIG. 8, the collate process is shown. In this, a loop 250/254/256/258 traverses the full set of records related to each patient 7, generating a health index of help needed for any of the patients 7. Note it is entirely possible that none of the patients 7 need help during the collate process. It is also possible that all patients 7 that need help have already been allocated resources 104A-104N. The collate process starts 250 with addressing the first patient 7 (U<-U₀). Now a loop begins (for each patient 7 . . . ), generating 254 a health index for the current patient 7, e.g., U. As discussed, the health index is any comparable value such as a numeric value, high/medium/low, etc. A test is made to determine if this is the last patient 256 and if not the last patient 256, the U is set to the next patient 258 and the loop continues. If U is already at the last patient 256, the allocation process runs.

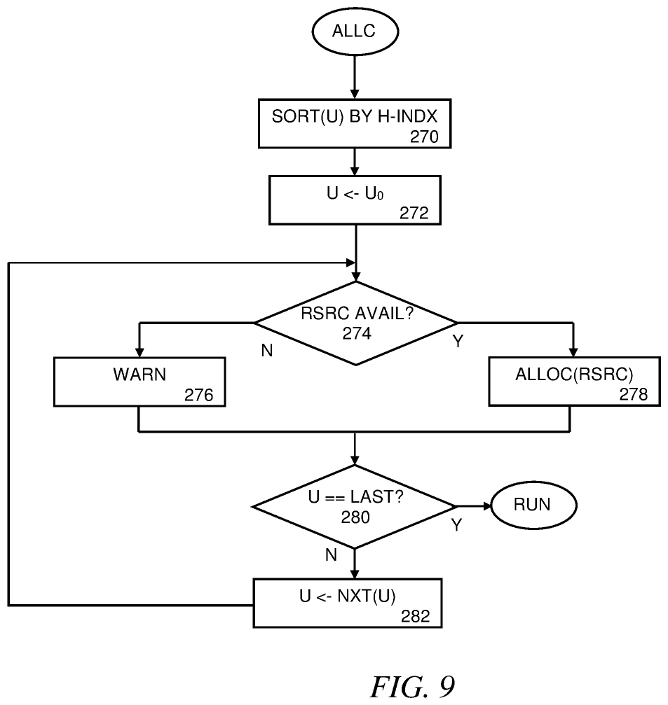

In FIG. 9, the allocation process is described. Note that for clarity and brevity reasons, the example allocation process is described without worry of double allocation such as allocating a resource 104A-104N to a patient 7 that has already been allocated a resource 104A-104N, though provisions to prevent double allocation is fully anticipated. Additionally, for the same reasons, the type of resource and other attributes are ignored by the sample allocation process, assuming the simplest model of, for example, an assisted care facility in which the patients 7 are patients of the facility and the resources 104A-104N are interchangeable (e.g., similar skill sets) or other types of staff that are equally interchangeable. In some other embodiments, the needs of the patient 7 vary as well as the skill sets of the resource 104A-104N. For example, the patient 7 might need cardiac assistance and only one of the resources 104A-104N has cardiac training. In such other embodiments, the resources 104A-104N are categorized by skills and those with specialized skills are recommended/allocated/dispatched to patients 7 in need of such skills.

The allocation process starts by sorting 270 the patients 7 by the health index, therefore, the patients 7 that have the worst current health (e.g., the highest health index) from the collate process migrate to the top of the patient list. Once sorted, the first patient 7 of the list is addressed 272 (U-<U₀)—the patient 7 having the worst current health (e.g., the highest health index). Now, in the allocation loop, it is determined if a resource 104A-104N is available 274 to help the current patient (e.g., is there another staff member that is free to help the current patient). In some embodiments, if no resource 104A-104N is available 274, then a warning is issued 276. Note, for simplicity, a warning (e.g., text message, writing to a log) is made each time a resource 104A-104N is needed and there are no resources available. It is fully anticipated that such warnings be made only when the health index of the current patient is a certain level. For example, warnings are only issued when the health index is high or greater than a certain numeric value. If, instead, a resource 104A-104N is available 274, a resource 104A-104N is allocated 278 to the current patient. Note, as above, if a resource 104A-104N was already allocated to the current patient, it is anticipated that a test be made to prevent double allocation.

In either case, a test 280 is then made to determine if the current patient is the last patient and, if so, the run processes is resumed. It the test 280 indicates that the current patient is not the last patient, the next patient 7 is addressed 282, and the allocation process continues.

In some embodiments of the system for resource allocation, instead of directly allocating resources 104A-104N, a status display is provided showing which patients are in need of resources. For example, a display showing all beds of a given facility with colors shading each bed, red for in dire need of a resource 104A-104N, yellow for in need of a resource 104A-104N, and green for no need of a resource 104A-104N. Given some sort of user interface like this, healthcare workers visually see where the needs exist and self-dispatch. Further, in some embodiments, for each patient 7 that is color coded red or yellow, an indicator (e.g., a letter) designates the type of resource 104A-104N needed (e.g., N for Nurse, D for doctor, C for cardiac, etc.).

It is believed that the system and method as described and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A system for resource allocation, the system comprising:
a plurality of patient locations for a patient;
a plurality of sensors at each of the patient locations, each sensor measuring health data of the patient when the patient is at the patient location;
an artificial intelligence engine, the artificial intelligence engine inputs the health data from the plurality of sensors over a period of time and calculates a base-health index for each of the patients, the base-health index for each of the patients is stored in a medical record of each patient; and
after the artificial intelligence engine calculates the base-health index for each of the patients, the artificial intelligence engine periodically inputs the health data from the plurality of sensors and calculates a current-health index for each of the patients, and if a difference between the base-health index for the patient and the current-health index of the medical record of the patient exceeds a threshold, the artificial intelligence engine issues an alarm; the system for resource allocation then sorts the patients by the current-health index for the patients to find the patients with a worst current-health index and initiates an action regarding each of the patients having the current-health index that is worse than an expected value.

2. The system of claim 1, wherein the action comprises dispatching a resource to the patient location.

3. The system of claim 1, wherein the action comprises recommending a resource to care for the patient location.

4. The system of claim 1, wherein the action comprises changing a display associated with the patient location on a patient monitoring system.

5. The system of claim 1, wherein each sensor of the plurality of sensors is selected from the group consisting of body temperature sensors, heart rate sensors, oxygen sensors, blood pressure sensors, skin color sensors, sleep sensors, toilet flush sensors, refrigerator opening sensors, pedometers, phone usage sensors, cameras, and microphones.

6. A method of resource allocation, the method comprising:
receiving, at an artificial intelligence engine, health data from sensors over a period, the sensors providing the health data of a plurality of patients, the artificial intelligence engine learning a baseline status of each patient in the plurality of patients during the period of time in a knowledge base;
after the period of time elapses, the artificial intelligence engine continuously receiving the health data from the sensors and when the health data singularly or in combination indicates an immediate health issue exists for one of the plurality of patients, the artificial intelligence engine allocating at least one resource to the one of the plurality patients associated with the health data that indicates the immediate health issue exists; and
periodically, the artificial intelligence engine scanning all health data from each of the plurality of patients and generating a health index for each patient in the plurality of patients using the knowledge base, then allocating/recommending at least one resource to a subset of the plurality of patients based upon the patients in the plurality of patients having a highest health index.

7. The method of claim 6, wherein the sensors are selected from the group consisting of body temperature sensors, heart rate sensors, oxygen sensors, blood pressure sensors, skin color sensors, sleep sensors, toilet flush sensors, refrigerator opening sensors, pedometers, phone input sensors, cameras, and microphones.

8. The method of claim 6, wherein the step of allocating/recommending the at least one resource to the subset of the plurality of patients based upon the patients in the plurality of patients having the highest health index further comprises dispatching a resource to the patient.

9. The method of claim 6, wherein the step of allocating the at least one resource to the subset of the plurality of patients based upon the patients in the plurality of patients having the highest health index further comprises changing a display associated with the patient on a patient monitoring system.

10. The method of claim 6, wherein after allocating/recommending the at least one resource, when a patient interaction is complete, capturing data from the patient interaction and inputting the data into the artificial intelligence engine and the artificial intelligence engine updates the knowledge base to make better future decisions.

11. The method of claim 6, after the step of periodically, the artificial intelligence engine scanning all health data from each of the plurality of patients, the artificial intelligence engine updates the knowledge base.

12. A computer-based system for resource allocation, the computer-based system comprising:
a computer;
a plurality of sensors that are electrically interfaced to the computer, each sensor measuring health data related to a patient within a population of patients;
an artificial intelligence engine interfaced to the computer, the artificial intelligence engine inputs the health data from the plurality of sensors over a period of time, generates a knowledge base using the health data, and calculates a base-health index for each of the patients, the base-health index for each of the patients is stored in a medical record of each patient; and
after the artificial intelligence engine calculates the base-health index for each of the patients, the artificial intelligence engine periodically inputs the health data from the plurality of sensors and calculates a current-health index for each of the patients, and if a difference between the base-health index for the patient and the current-health index of the medical record of the patient exceeds a threshold, the artificial intelligence engine issues an alarm; the system for resource allocation then sorts the patients by the current-health index for the patients to find the patients with a worst current-health index and initiates an action regarding each of the patients having the current-health index that is worse than an expected value.

13. The computer-based system of claim 12, wherein the action comprises recommending allocation of a resource to the patient.

14. The computer-based system of claim 12, wherein the action comprises dispatching a resource to the patient.

15. The computer-based system of claim 12, wherein the action comprises changing a display associated with the patient on a patient monitoring system.

16. The computer-based system of claim 12, wherein the sensors are electrically interfaced to the computer through a data network.

17. The computer-based system of claim 12, wherein when the artificial intelligence engine periodically inputs the health data from the plurality of sensors and calculates the current-health index for each of the patients, the artificial intelligence engine updates the knowledge base.

18. The computer-based system of claim 12, wherein when the action is complete, data from a patient interaction is captured and entered into the artificial intelligence engine and the artificial intelligence engine updates the knowledge base to make better future decisions.

19. The computer-based system of claim 12, wherein the plurality of sensors are selected from the group consisting of body temperature sensors, heart rate sensors, oxygen sensors, blood pressure sensors, skin color sensors, sleep sensors, toilet flush sensors, refrigerator opening sensors, pedometers, phone usage sensors, cameras, and microphones.
